(19) United States
(12) Patent Application Publication
(10) Pub. No.: US 2022/0184187 A1
(43) Pub. Date: Jun. 16, 2022

(54) Combination treatment for atopic dermatitis

(71) Applicant: Micreos Human Health B.V., Den Haag (NL)

(72) Inventors: Mark Leonard Offerhaus, Den Haag (NL); Fritz Eichenseher, Zürich (CH); Martin Johannes Loessner, Ebmatingen (CH)

(73) Assignee: Micreos Human Health B.V., Den Haag (NL)

(21) Appl. No.: 17/687,789

(22) Filed: Mar. 7, 2022

Related U.S. Application Data
(62) Division of application No. 16/439,734, filed on Jun. 13, 2019.
Application No. 16/439,734 is a division of application No. 14/904,387, filed on Jan. 11, 2016, now Pat. No. 10,357,547.

(30) Foreign Application Priority Data
Jul. 11, 2013 (EP) 13176034.0

(51) Int. Cl. A61K 38/47; A61K 38/16; A61K 38/21; A61K 31/573; A61K 31/56; A61K 45/06

(52) U.S. Cl. CPC A61K 38/47; A61K 38/162; A61K 38/217; C12Y 302/01017; A61K 31/56; A61K 45/06; A61K 31/573

(57) ABSTRACT

The invention relates to the field of medicine, specifically to the field of treatment of dermatitis or eczema, even more specifically to the field of treatment of atopic dermatitis. The invention relates to a novel composition and a novel kit of parts, both comprising an anti-inflammatory compound and a compound specifically targeting a bacterial cell, preferably a gram positive bacterial cell. The invention further relates to said composition and/or kit of parts for medical use, preferably for treating an individual suffering from eczema.

### FIELD OF THE INVENTION

The invention relates to the field of medicine, specifically to the field of treatment of dermatitis or eczema, even more specifically to the field of treatment of atopic dermatitis. The invention relates to a novel composition and a novel kit of parts, both comprising an anti-inflammatory compound and a compound specifically targeting a bacterial cell, preferably a gram positive bacterial cell. The invention further relates to said composition and/or kit of parts for medical use, preferably for treating an individual suffering from eczema.

### BACKGROUND OF THE INVENTION

Eczema is a common skin condition characterized by red, itchy skin and small blisters also known in the art as skin rash or rash or dermatitis. There is no cure for eczema, but there are many treatments, ranging from special diets to emollients and immunosuppressive ointments like e.g. corticosteroid ointment. While corticosteroids, such as hydrocortisone or clobetasol propionate (topical, oral or intradermal administration) usually bring about improvements, they also may have side effects. Prolonged use of topical corticosteroids is thought to increase the risk of side effects, the most common of which is the skin becoming thin and fragile (atrophy). Because of this, if used on the face or other delicate skin, a low-strength steroid should be used or applied less frequently. Additionally, high-strength steroids used over large areas, or under occlusion, may be absorbed into the body, causing hypothalamic-pituitary-adrenal axis suppression (HPA axis suppression).

Due to the impaired skin barrier in atopic dermatitis an increase in skin infections with bacteria such as Staphylococcus aureus or fungi might be the result. For more severe cases, dermatologists may also prescribe either topical or oral conventional antibiotics such as penicillin, streptomycin and chloramphenicol. The antibiotics prevent infection that can result from impaired skin barrier such as cracked skin. S. aureus colonization or infection is the most common cause of increased eczema severity. The effectiveness of antibiotic treatments varies from person to person. The well known disadvantages of conventional antibiotics are a-specificity, i.e. also non-pathogenic and/or beneficial bacteria are killed, and the risk of developing resistance, not only by the target bacterial cells but possibly also by other pathogenic bacteria. Furthermore, conventional, systemic antibiotic treatment can interact with other drugs, including contraceptive pills. Certain antibiotics cannot be combined with the use of alcohol. Accordingly, there is a need for improved treatment of eczema.

### DESCRIPTION OF THE INVENTION

In a first aspect, the present invention provides for a novel composition comprising a first and a second compound, wherein said first compound is an anti-inflammatory compound and said second compound is a compound specifically targeting a bacterial cell, preferably a gram positive bacterial cell. Preferably, said gram positive bacterial cell is a Staphylococcus, more preferably a Staphylococcus aureus. Preferably, said composition is a medicament preferably for use in the treatment of eczema, most preferably for use in the treatment of atopic dermatitis, as further detailed herein. The state of the art for treating eczema is using immunosuppressive agents like corticosteroids and if indicated antibiotics topical or systemic. The present invention provides for a novel composition comprising both an anti-inflammatory compound and a compound specifically targeting a bacterial cell, preferably a gram positive bacterial cell, preferably a Staphylococcus aureus, which combats most, if not all, of the disadvantages of using either an effective dosage regime of a corticosteroid and/or a conventional antibiotic alone or in combination and provides a unexpected synergy. In comparison to the use of an immunosuppressive agent alone like a corticosteroid alone, a composition of the invention decreases the risk and/or is more effective by combating eczema-related and/or corticosteroid induced infections induced by a bacterial cell, such as a gram positive bacterial cell, preferably Staphylococcus aureus. Furthermore, in comparison to the use of a corticosteroid alone, a composition according to the invention may be as effective as using a corticosteroid alone while making use of a lower dosage and/or a shorter administration regimen resulting in a shorter exposure time of the corticosteroid thereby reducing possible side-effects like the risk of skin atrophy, hypothalamic-pituitary-adrenal axis suppression (HPA axis suppression) and/or (increased) skin infection.

In comparison to the use of a corticosteroid in combination with conventional antibiotics and/or conventional antibiotics alone, the composition of the present invention selectively specifically targets a bacterial cell, preferably a gram positive bacterial cell, preferably a Staphylococcus, more preferably a Staphylococcus aureus, without affecting surrounding commensal and/or beneficial microflora. In addition, the risk of developing resistance against antibiotics is diminished or at least reduced since lower amounts of antibiotics or even no antibiotics at all are used.

An agent that specifically targets a gram positive bacterial cell preferably is an agent that shows at least 2, 5, 10, 50 or 100 times higher lytic activity towards a gram positive bacterial cell as compared to a gram negative bacterial cell. Preferably, an agent that specifically targets a gram positive bacterial cell is an agent that does not affect a gram negative bacterial cell in a concentration that is affective in lysing a gram positive bacterial cell. An agent that specifically targets a Staphylococcus bacterial cell preferably is an agent that shows at least 2, 5, 10, 50 or 100 times higher lytic activity towards a Staphylococcus bacterial cell as compared to a non-Staphylococcus bacterial cell. Preferably, an agent that specifically targets a Staphylococcus bacterial cell is an agent that does not affect a non-Staphylococcus bacterial cell in a concentration that is effective in lysing a Staphylococcus bacterial cell. An agent that specifically targets a Staphylococcus aureus bacterial cell preferably is an agent that shows at least 2, 5, 10, 50 or 100 times higher lytic activity towards a Staphylococcus aureus bacterial cell as compared to a non-Staphylococcus aureus bacterial cell. Preferably, an agent that specifically targets a Staphylococcus aureus bacterial cell is an agent that does not affect a non-Staphylococcus aureus bacterial cell in a concentration that is effective in lysing a Staphylococcus aureus bacterial cell. Lytic activity is preferably assessed as exemplified herein.

Preferably, the invention provides a composition comprising a first and a second compound, wherein said first compound is an anti-inflammatory compound and said second compound is a compound specifically targeting a bacterial cell, preferably a gram positive bacterial cell, and wherein said second compound comprises at least one cell wall binding domain specifically binding the peptidoglycan cell wall of said bacterial cell, preferably gram positive bacterial cell. A cell wall-binding domain of the present invention is defined as an element, preferably a polypeptide within said second compound that directs said second compound to the bacterial wall of a bacterial cell.

A cell wall-binding domain encompassed within the present invention may be any cell wall-binding domain known by the person skilled in the art. Preferably, a cell wall-binding domain of the present invention is an element, preferably a polypeptide within said second compound, that directs said second compound to the peptidoglycan cell wall of a gram-positive bacterial cell, preferably the peptidoglycan cell wall of a Staphylococcus bacterial cell, more preferably the peptidoglycan cell wall of a Staphylococcus aureus bacterial cell.

Preferably, the invention provides a composition comprising a first and a second compound, wherein said first compound is an anti-inflammatory compound and said second compound is a compound specifically targeting a bacterial cell, preferably a gram positive bacterial cell, and wherein said second compound comprises at least one cell wall binding domain specifically binding the peptidoglycan cell wall of Staphylococcus, more preferably, a Staphylococcus aureus.

Binding of a domain to the peptidoglycan cell wall of Staphylococcus genera may be assessed using assays well known to the person skilled in the art. In a preferred embodiment, an immunohistochemical technique and/or a gene fusion technique resulting in labelled constructs are used for assessing specific binding of compounds such as peptides, polypeptides, proteins or bacteriophages to the peptidoglycan cell wall of Staphylococcus genera. Quantification methods of signals used in the above mentioned immunohistochemical or fusion techniques are well known in the art.

In one embodiment, Staphylococcus peptidoglycan cell wall-binding is quantified using a fluorescent fusion construct comprising a cell wall-domain of interest. Such a cell wall-binding assay is described in detail by Loessner et al (Molecular Microbiology 2002, 44(2): 335-349). In this assay a solution comprising said fluorescent fusion construct or a negative control, preferably Green Fluorescent Protein (GFP), is subjected to Staphylococcus cells, preferably S. aureus cells, more preferably S. aureus BB255 for an indicated time period where after the cells are sedimented by centrifugation together with the bound fluorescent fusion constructs. The fluorescent signal of the Staphylococcus cells exposed to a fluorescent fusion construct subtracted by the fluorescence signal of the Staphylococcus cells exposed to a negative control, preferably GFP, is a measure for cell binding as meant in this disclosure. Preferably, within the context of the invention, a domain is said to bind the peptidoglycan cell wall of Staphylococcus genera when using this assay an increase in fluorescent signal of the sedimented cells above the negative control as defined herein is detected. Preferably, the invention relates to a cell wall-binding domain which exhibits binding as defined herein of at least 50, 60, 70, 80, 90 or 100, 150 or 200% of peptidoglycan cell wall-binding of S. aureus bacteriophage Φ2638a endolysin (Ply2638 endolysin defined by SEQ ID NO: 2) preferably encoded by SEQ ID NO: 1. Preferably, a fusion construct as represented by SEQ ID NO: 95 and encoded by SEQ ID NO: 96 serves as a positive control in this assay. An overview of all sequences included and their SEQ ID NO is given in table 2.

Preferably, the invention provides a composition comprising a first and a second compound, wherein said first compound is an anti-inflammatory compound and said second compound is a compound specifically targeting a bacterial cell, preferably a gram positive bacterial cell, and wherein said second compound comprises at least one cell wall binding domain that originates from or is a homologue of a Staphylococcus phage endolysin, preferably said Staphylococcus phage endolysin is selected from, but not limited to, S. aureus bacteriophage Φ2638a endolysin, S. aureus bacteriophage Φ11 endolysin, S. aureus bacteriophage ΦTwort endolysin, S. haemolyticus JCSC1435, S. aureus Phage K endolysin, S. warneri phage WMY endolysin, S. aureus phage NM3 endolysin and S. aureus 80alpha endolysin.

Also preferred is a cell wall binding domain originating from or a homologue of S. simulans lysostaphin (represented by SEQ ID NO: 76, preferably encoded by SEQ ID NO: 75). A known homologue of S. simulans lysostaphin having cell wall binding properties is S. capitis ALE-1 enzyme.

Preferably, said cell wall binding domain has at least 80% identity to any of SEQ ID NO: 4, 6 or 8 and/or wherein said one or more enzymatic active domains has at least 80% identity to any of SEQ ID NO: 10, 12, 14, 16, 18, 98 or 100. A preferred cell wall-binding domain of the present invention is a cell wall-binding domain having at least 80, 81, 82, 83, 84, 85, 86, 87, 88, 89, 90, 91, 92, 93, 94, 95, 96, 97, 98, 99 or 100% identity with the cell wall binding domain of S. simulans lysostaphin defined herein by SEQ ID NO: 4 and preferably encoded by SEQ ID NO: 3. Also preferred is a cell wall-binding domain isolated from a native Staphylococcus bacteriophage endolysin. Also preferred is a cell wall-binding domain of the present invention that has at least 80, 81, 82, 83, 84, 85, 86, 87, 88, 89, 90, 91, 92, 93, 94, 95, 96, 97, 98, 99 or 100% identity with the cell wall-binding domain of S. aureus bacteriophage Φ2638a endolysin defined herein by SEQ ID NO: 6 and preferably encoded by SEQ ID NO: 5. Also preferred is a cell wall-binding domain isolated from a native Staphylococcus aureus phage phiNM3 endolysin. Preferably, a cell wall-binding domain of the present invention has at least 80, 81, 82, 83, 84, 85, 86, 87, 88, 89, 90, 91, 92, 93, 94, 95, 96, 97, 98, 99 or 100% identity with the cell wall-binding domain of S. aureus phage phiNM3 endolysin defined herein by SEQ ID NO: 8 and preferably encoded by SEQ ID NO: 7.

Preferably, the invention provides for a composition comprising a first and a second compound, wherein said first compound is an anti-inflammatory compound and said second compound is a compound specifically targeting a bacterial cell, preferably a gram positive bacterial cell, wherein said second compound comprises one or more enzymatic active domains exhibiting target bond specificity. ‘An enzymatic active domain’ is defined herein is a domain having lytic activity, preferably exhibiting peptidoglycan hydrolase activity. Lytic activity can be assessed by methods well known by the person skilled in the art. In an embodiment, lytic activity is assessed spectrophotometrically by measuring the drop in turbidity of substrate cell suspensions. Turbidity is assessed by measuring optical density at a wavelength of 595 nm, typically a culture as turbid when it exhibits an optical density of at least 0.3 OD at a wavelength of 595 nm. Preferably, lytic activity is assessed spectrophotometrically measuring the drop in turbidity of a S. aureus suspension, wherein turbidity is quantified by measuring OD₅₉₅ spectrophotometrically (Libra S22, Biochrom). More preferably, 200 nM polypeptide comprising an enzymatic active domain of the invention as identified herein is incubated together with an S. aureus suspension having an initial OD₅₉₅ of 1±0.05, as assessed spectrophotometrically (Libra S22, Biochrom), in PBS buffer pH 7.4, 120 mM sodium chloride for 30 min at 37° C. The drop in turbidity is calculated by subtracting the OD₅₉₅ after 30 min of incubation from the OD₅₉₅ before 30 min of incubation. Within the context of the invention a polypeptide comprising an enzymatic active domain of the invention as identified herein will be said to have lytic activity if, when using this assay, a drop in turbidity of at least 10, 20, 30, 40, 50 or 60% is detected. Preferably, a drop in turbidity of at least 70% is detected. Preferably, a polypeptide comprising an enzymatic active domain of the invention exhibits a lytic activity of at least 30, 40, 50, 60, 70, 80, 90, 100, 150 or 200% or more of a lytic activity of S. aureus bacteriophage Φ2638a endolysin (Ply2638 endolysin identified by SEQ ID NO: 2) preferably encoded by SEQ ID NO: 1.

Preferably, the invention provides for a composition comprising a first and a second compound, wherein said first compound is an anti-inflammatory compound and said second compound is a compound specifically targeting a bacterial cell, preferably a gram positive bacterial cell, wherein said second compound comprises one or more enzymatic active domains exhibiting target bond specificity, and wherein said target bond is an essential bond in a peptidoglycan layer of said bacterial cell, preferably gram positive bacterial cell. An essential bond in a peptidoglycan layer of a bacterial cell, preferably a gram-positive bacterial cell is defined herein as a linkage within said peptidoglycan that is essential for said peptidoglycan to provide said bacterial cell shape and a rigid structure resistance to osmotic shock. Preferably, said essential bond in a peptidoglycan layer of a gram-positive bacterial cell is a bond between a D-alanine of the stem peptide and a glycine of the cross-bridge peptide (defined herein also as a bond between an N-terminal alanine and a glycine), a bond in a pentaglycin cross-bridge (defined herein also as a pentyglycin bridge glycyl-glycyl bond, a bond between an N-acetylmuramoyl and an L-alanine or a bond between an N-acetylmuramine and a N-acetylglucosamine or between a N-acetlyglucosamine and an N-acetylmuramine. Other preferred essential bonds in a peptidoglycan layer of a gram-positive bacterial cell are a bond in a gamma-glutamyl stem peptide, a bond between an L-alanyl-iso-D-glutamic acid in a stem peptide and a bond between an iso-D-glutamic acid-L-Lysine in a stem peptide.

Most native Staphylococcus bacteriophage endolysins exhibiting peptidoglycan hydrolase activity consist of a C-terminal cell wall-binding domain (CBD), a central N-acetylmuramoyl-L-Alanine amidase domain, and an N-terminal alanyl-glycyl endopeptidase domain with cysteine, histidine-dependent amidohydrolases/peptidase (CHAP) homology, or in case of Ply2638, of an N-terminal glycyl-glycine endopeptidase domain with Peptidase_M23 homology, the latter three domains exhibiting peptidoglycan hydrolase activity each with distinct target bond specificity and generally named herein as enzymatically active domains. Preferably, said one or more enzymatic active domains is selected from or is a permutation of a domain of the group consisting of a cysteine, histidine dependent amidohydrolases/peptidase domain, an endopeptidase domain, an amidase domain and a glycosylhydrolase domain. Said glycosylhydrolase domain can be a muramidase domain or a glycosaminidase domain.

Preferably, said CHAP domain cleaves a bond between an N-terminal alanyl and a glycyl within a peptidoglycan layer. More preferably, said CHAP domain specifically cleaves a bond between an N-terminal alanyl and a glycyl within a peptidoglycan layer. Preferably, said endopeptidase domain cleaves pentaglycin bridge glycyl-glycyl bond within a peptidoglycan layer. More preferably, said endopeptidase domain specifically cleaves pentaglycin bridge glycyl-glycyl bond within a peptidoglycan layer. Preferably, said amidase domain cleaves a bond between a central N-acetlymuramoyl and an L-Alanine within a peptidoglycan layer. More preferably, said amidase domain specifically cleaves a bond between a central N-acetlymuramoyl and an L-Alanine within a peptidoglycan layer. Preferably, said murimidase domain cleaves a bond between an N-acetylmuramine and a N-acetylglucosamine within a peptidoglycan layer. More preferably, said murimidase domain specifically cleaves a bond between an N-acetylmuramine and a N-acetylglucosamine within a peptidoglycan layer. Preferably, said glucosaminidase domain cleaves a bond between an N-acetlyglucosamine and an N-acetylmuramine within a peptidoglycan layer. More preferably, said glucosaminidase domain specifically cleaves a bond between an N-acetlyglucosamine and an N-acetylmuramine within a peptidoglycan layer. Preferably said peptidoglycan layer is of a bacterial cell, preferably a gram positive bacterial cell, more preferably of a Staphylococcus, most preferably of a Staphylococcus aureus. Preferably, the cleavage of a bond by an enzymatic active domain as defined herein is specific if such a bond is hydrolysed at least 2, 5, 10, 50 or a 100 times more efficient with said enzymatic active domain as compared to the hydrolyses of any other bond as defined herein above with said enzymatic active domain.

Preferably, a CHAP domain encompassed within the present invention originates from Staphylococcus phage K, Staphylococcus phage Twort and/or S. aureus bacteriophage phi 11. Preferably, a CHAP domain encompassed within the present invention, is a domain that has at least 80, 81, 82, 83, 84, 85, 86, 87, 88, 89, 90, 91, 92, 93, 94, 95, 96, 97, 98, 99 or 100% identity with SEQ ID NO: 10, 12 or 98 and/or is preferably encoded by SEQ ID NO: 9 or 11. Preferably, an endopeptidase domain encompassed within the present invention originates from S. aureus bacteriophage Φ2638a and/or S. simulans. Preferably, an endopeptidase domain encompassed by the present invention has at least 80, 81, 82, 83, 84, 85, 86, 87, 88, 89, 90, 91, 92, 93, 94, 95, 96, 97, 98, 99 or 100% identity with SEQ ID NO: 14 or 16 and/or is preferably encoded by SEQ ID NO: 13 or 15. Preferably, an amidase domain encompassed within the present invention originates from S. aureus bacteriophage Φ2638a or S. aureus bacteriophage phi 11. Preferably an amidase domain of the present invention has at least 80, 81, 82, 83, 84, 85, 86, 87, 88, 89, 90, 91, 92, 93, 94, 95, 96, 97, 98, 99 or 100% identity with SEQ ID NO: 18 or 100 and/or is preferably encoded by SEQ ID NO: 17 or 99.

Preferably, the invention provides for a composition comprising a first and a second compound, wherein said first compound is an anti-inflammatory compound and said second compound is a compound specifically targeting a bacterial cell, preferably a gram positive bacterial cell, wherein said second compound is a naturally occurring or mutant bacteriophage, a naturally occurring endolysin or a mutant polypeptide.

A naturally occurring bacteriophage of the present invention may be any bacteriophage specifically targeting and infecting a a bacterial cell, preferably bacterial cell, preferably a Staphylococcus, most preferably a Staphylococcus aureus. Preferably, a naturally occurring bacteriophage of the present invention is selected from, but not limited to, a group consisting of S. aureus bacteriophage Φ2638a, S. aureus bacteriophage Φ11, S. aureus bacteriophage ΦTwort, S. haemolyticus JCSC 1435, S. aureus Phage K, S. warneri phage WMY, S. aureus phage NM3 and S. aureus 80alpha. Said naturally occurring endolysin may be synthesized and/or purified. A bacteriophage according to the invention may be a mutant, chimeric and/or recombinant bacteriophage. The person skilled in the art may construct a bacteriophage of the present invention by placing mutations in the genome and/or deleting and/or inserting coding sequences or parts thereof into the genome using methods known in the art.

A naturally occurring endolysin may be any wild type or native endolysin exhibiting peptidoglycan hydrolase activity. Preferred is a Staphylococcus phage endolysin, preferably said Staphylococcus phage endolysin is selected from, but not limited to, the group consisting of S. aureus bacteriophage Φ2638a endolysin, S. aureus bacteriophage Φ11 endolysin, S. aureus bacteriophage ΦTwort endolysin, S. haemolyticus JCSC1435, S. aureus Phage K endolysin, S. warneri phage WMY endolysin, S. aureus phage NM3 endolysin and S. aureus 80alpha endolysin. Also preferred is S. simulans lysostaphin and/or a homologue of S. simulans lysostaphin such as S. capitis ALE-1 enzyme. Most native Staphylococcus bacteriophage endolysins exhibiting peptidoglycan hydrolase activity consist of a C-terminal cell wall-binding domain (CBD), a central N-acetylmuramoyl-L-Alanine amidase domain, and an N-terminal Alanyl-glycyl endopeptidase domain with CHAP homology, or in case of Ply2638, of an N-terminal endopeptidase domain with Peptidase_M23 homology, the latter three domains exhibiting peptidoglycan hydrolase activity each with distinct target bond specificity and generally named herein as enzymatically active domains.

A mutant polypeptide as encompassed within the present invention may be a chemically synthesized polypeptide or a recombinant or retrofitted polypeptide produced in vitro. A retrofitted construct is defined herein as a polynucleotide comprising heterologous nucleotide sequences. As used herein the term heterologous sequence or heterologous polynucleotide is one that is not naturally found operably linked as neighboring sequence of said first nucleotide sequence. As used herein, the term heterologous may mean recombinant. Recombinant refers to a genetic entity distinct from that generally found in nature. As applied to a nucleotide sequence or nucleic acid molecule, this means that said nucleotide sequence or nucleic acid molecule is the product of various combinations of cloning, restriction and/or ligation steps, and other procedures that result in the production of a construct that is distinct from a sequence or molecule found in nature. Preferably, a mutant polypeptide to the present invention comprises at least an enzymatic active domain and a cell binding domain as defined herein.

An endolysin or mutant polypeptide of the present invention may be in a purified form or may be comprised within a crude composition, preferably of biological origin, such as a bacterial lysate, yeast lysate, fungal lysate, sonicate or fixate. Alternatively, said endolysin or mutant polypeptide may be a chemically synthesized endolysin or polypeptide or a recombinant polypeptide produced in vitro.

An endolysin or mutant polypeptide of the present invention preferably comprises or consists of at least one enzymatic active domain and at least one cell binding domain and optionally a tag for ease of purification. Preferably, said tag is selected from, but is not limited to, the group consisting of a FLAG-tag, poly(His)-tag, HA-tag and Myc-tag. More preferably said tag is a 6× His-tag. Even more preferably, said tag is an N-terminal 6× His-tag (indicated herein as HXa) identical to SEQ ID NO: 74 and preferably encoded by SEQ ID NO: 73).

Preferably, a cell wall-binding domain according to the present invention is located on the C-terminal side of the enzymatic active domain within said naturally occurring endolysin or a mutant polypeptide. Preferably, said mutant naturally occurring or mutant polypeptide comprises at least two or more enzymatic active domains with distinct target bond specificities as distinct target bond specificities confer synergistic effects. In an embodiment of the invention, a composition comprises at least two distinct compounds targeting a bacterial cell, preferably a gram positive bacterial cell, preferably a Staphylococcus, more preferably a Staphylococcus aureus. Preferably said at least two distinct compounds are naturally occurring endolysin, which are optionally synthesized. Preferably said at least two distinct compounds are recombinant polypeptides each comprising a distinct enzymatic active domain and/or a different multiplicity of at least two distinct enzymatic active domains as defined herein below.

Preferably, the invention provides for a composition comprising a first and a second compound, wherein said first compound is an anti-inflammatory compound and said second compound is a compound specifically targeting a bacterial cell, preferably a gram positive bacterial cell, wherein said second compound is a recombinant polypeptide comprising a multiplicity of said one or more enzymatic active domains exhibiting target bond specificity. “Multiplicity” is to be understood as a number of copies and may be any integer varying from 1 to 20, preferably from 1 to 10, more preferably from 1 to 3, most preferably said multiplicity is 2, i.e. a duplicate. Polypeptides comprising a multiplicity of enzymatic active domains show superior lytic activity as compared to polypeptides comprising a single enzymatic active domain.

Preferably, said second compound is a polypeptide comprising and/or consisting of an enzymatic active domain, a cell wall binding and optionally a tag for ease of purification as defined herein, preferably said enzymatic active domain being a cysteine, histidine-dependent amidohydrolases/peptidase domain, an endopeptidase domain or an amidase domain, and preferably polypeptide comprises a multiplicity of said enzymatic active domain, preferably said multiplicity being 2, i.e. a duplicate. More preferably said polypeptide comprises and/or consists of a duplicated amidase domain and a cell wall binding domain and optionally a tag for ease of purification as defined herein, preferably said amidase is from S. aureus bacteriophage Φ2638a endolysin and said cell wall binding domain is of S. simulans lysostaphin. Most preferably said polypeptide comprises and/or consists of a duplicated endopeptidase domain and a cell wall binding domain and optionally a tag for ease of purification as defined herein, preferably said endopeptidase domain is a Peptidase_M23 domain of S. simulans lysostaphin and said cell wall binding domain is of S. simulans lysostaphin.

Preferably, said second compound is a polypeptide has at least 80, 81, 82, 83, 84, 85, 86, 87, 88, 89, 90, 91, 92, 93, 94, 95, 96, 97, 98, 99 or 100% identity with SEQ ID NO: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 76, 78, 80, 82, 84, 86, 88, 90, 92, 94, 98 or 100 and/or is encoded by a polynucleotide having at least 80, 81, 82, 83, 84, 85, 86, 87, 88, 89, 90, 91, 92, 93, 94, 95, 96, 97, 98, 99 or 100% identity to any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 75, 77, 79, 81, 83, 85, 87, 89, 91, 93, 97 or 99. Preferably, said polypeptide has at least 80, 81, 82, 83, 84, 85, 86, 87, 88, 89, 90, 91, 92, 93, 94, 95, 96, 97, 98, 99 or 100% identity with SEQ ID NO: 28, 34, 46, 52, 58 or 70, more preferably, said polypeptide has at least 80, 81, 82, 83, 84, 85, 86, 87, 88, 89, 90, 91, 92, 93, 94, 95, 96, 97, 98, 99 or 100% identity with SEQ ID NO: 28, 46, 52, or 70, even more preferably, said polypeptide has at least 80, 81, 82, 83, 84, 85, 86, 87, 88, 89, 90, 91, 92, 93, 94, 95, 96, 97, 98, 99 or 100% identity with SEQ ID NO: 46 or 70, most preferably said polypeptide has at least 80, 81, 82, 83, 84, 85, 86, 87, 88, 89, 90, 91, 92, 93, 94, 95, 96, 97, 98, 99 or 100% identity with SEQ ID NO: 70.

Preferably, the invention provides for a composition comprising a first and a second compound, wherein said first compound is an anti-inflammatory compound and said second compound is a compound specifically targeting a bacterial cell, preferably a gram positive bacterial cell, wherein said first compound is selected from the group consisting of a corticosteroid, a calcineurin inhibitor, an immunotherapeutic compound, a recombinant human IFN-gamma, a microbial probiotic, a cytokine modulator, an inflammatory cell recruitment blocker, and a T cell activation inhibitor. Preferred calcineurin inhibitors are FK506, tacrolimus and pimecrolimus. A preferred immunotherapeutic compound is omalizumab, a humanized IgG1 monoclonal antibody against IgE that recognizes and masks an epitope in the CH3 region of IgE responsible for binding to the high-affinity FcεR on mast cells and basophils. Preferred cytokine modulators are a soluble IL-4 receptor, an anti-IL-5 monoclonal antibody and a TNF inhibitor. Preferred inflammatory cell recruitment blockers are a chemokine receptor antagonist and a CLA inhibitor. Preferred T cell activation inhibitors are alefacept and efalizumab. Corticosteroids that may be used include, but are not limited to, betamethasone dipropionate, fluocinolone acetonide, betamethasone valerate, triamcinolone acetonide, clobetasol propionate, desoximetasone, diflorasone diacetate, amcinonide, flurandrenolide, hydrocortisone valerate, hydrocortisone butyrate, desonide, hydrocortisone and methylprednisolone acetate. A further preferred anti-inflammatory compound is dapsone which has both antimicrobial and anti-inflammatory properties.

In all embodiments of the present invention, the second compound specifically targeting a bacterial cell, preferably a gram positive bacterial cell may be comprised of a combination of a source of a first enzymatic active domain and a source of a second enzymatic active domain, wherein said first and second enzymatic active domains exhibit distinct target bond specificities and are comprised on a distinct first and second polypeptide, i.e. said first enzymatic active domain is comprised on a first polypeptide and said second enzymatic domain is comprised on a second polypeptide, wherein said first and second polypeptide each have a distinct amino acid sequence. In addition, the second compound according to the present invention may be comprised of a combination of a source of a first enzymatic active domain, a source of a second enzymatic active domain and a source of a third enzymatic active domain, wherein said first, second and third enzymatic active domain exhibit distinct target bond specificities and are comprised on a distinct first, second and third polypeptide, i.e. said first enzymatic active domain is comprised on a first polypeptide, said second enzymatic domain is comprised on a second polypeptide, and said third enzymatic domain is comprised on a third polypeptide, wherein said first, second and third polypeptide each have a distinct amino acid sequence. Furthermore, the second compound according to the present invention may be comprised of a combination of a source of a first enzymatic active domain, a source of a second enzymatic active domain, a source of a third enzymatic active domain, and a source of a further enzymatic active domain, wherein said first, second, third and further enzymatic active domain exhibit distinct target bond specificities and are comprised on a distinct first, second, third and further polypeptide, i.e. said first enzymatic active domain is comprised on a first polypeptide, said second enzymatic domain is comprised on a second polypeptide, said third enzymatic domain is comprised on a third polypeptide, and said further enzymatic active domain is comprised on a further polypeptide, wherein said first, second, third and further polypeptide each have a distinct amino acid sequence. A further enzymatic active domain is meant herein as a fourth, fifth, sixth, seventh, eighth, ninth, tenth or more enzymatic active domain, preferably a fourth enzymatic active domain. A further polypeptide is meant herein as a fourth, fifth, sixth, seventh, eighth, ninth, tenth or more polypeptide, preferably a fourth polypeptide.

The inventors surprisingly found for the second compound according to the invention, that simultaneous application of two or more enzymatically active domains with distinct target bond specificities confers synergistic effects. Surprisingly, this works not only when enzymatically active domains with different specificities are located on the same molecule as in native Staphylococcus endolysins, but works also when the enzymatically active domains with different specificities are separated on distinct polypeptides.

The benefit of having distinct enzymatic active domains located on separate individual polypeptides is that the resulting polypeptides are smaller which can be more easily produced. Furthermore, these smaller polypeptides have better diffusion properties in specific environments and can be more resistant to degradation and feature higher thermostability. Another advantage is that independent distinct enzymatic active domains located on separate distinct polypeptide molecules can be mixed and pooled in variable compositions, at a ratio that is best suited to hydrolyse the specific bacterial target cells. The second compound according to the invention comprised of a combination as described herein can be supplemented and/or complemented by the use of virtually any functional enzymatic active domain with virtually any target bond specificity from many different origins including phage lysins, bacteriocins, autolysins, or any other cell wall lytic enzymes.

Within the context of the second compound according to the present invention ‘a combination’ means that a source of a first enzymatic active domain and a source of a second enzymatic active domain are contemplated and encompassed. In addition, within the context of the second compound according to present invention ‘a combination’ means that a source of a first enzymatic active domain, a source of a second enzymatic active domain and optionally a source of a third and/or further enzymatic active domain are contemplated and encompassed. Each source may be together or present together or combined together or physically in contact with the other source forming one single composition. Each source may alternatively be comprised within a distinct composition. However the present invention provides the insight that both sources of a first and a second enzymatic active domain are preferably needed or are used in order to get an effect of the present invention as defined herein. If each source is not present in a same single composition, each source and/or each distinct composition comprising a source of a combination encompassing the second compound according to the present invention may be used sequentially or simultaneously.

‘A source of a first enzymatic active domain’, ‘a source of a second enzymatic active domain’, ‘a source of a third enzymatic active domain’ and ‘a source of a further enzymatic active domain’ preferably comprises a protein-based source, i.e. a polypeptide, a protein, digest of a protein and/or fragment of a protein or digest, or a source not being protein based, i.e. a nucleic acid encoding a protein or derived peptide or protein fragment. Below we define preferred sources of a first enzymatic active domain, a source of a second enzymatic active domain, a source of a third enzymatic active domain and a source of a further enzymatic active domain that are encompassed by the invention. When the second compound according to the invention relates to a combination of a source of a first enzymatic active domain, a source of a second enzymatic active domain and optionally a source of a third and/or further enzymatic active domain, each of the sources of a first enzymatic active domain defined herein may be combined with each of the sources of a second and optionally third and/or further enzymatic active domain defined herein. It is also encompassed by the present invention to use a combination of a source of a first enzymatic active domain being protein-based with a source of a second and optionally a third and/or further enzymatic active domain being not protein-based, and vice versa.

‘Comprised on distinct polypeptides’ is meant herein as any of said first, second and optionally third and/or further enzymatic active domain is comprised on a polypeptide which is distinct from the polypeptide that any of the other of said first, second and optionally third and/or further enzymatic active domain is comprised on.

In all embodiments according to the invention, a polypeptide can be a natural polypeptide or an isolated polypeptide, preferably an isolated polypeptide. A nucleic acid according to the present invention may be a natural nucleic acid or an isolated nucleic acid, preferably an isolated nucleic acid. A nucleic acid construct according to the present invention can be a natural or an isolated construct, preferably an isolated nucleic acid construct.

Preferably, a first, a second and optionally a third and/or further enzymatic active domain together encompassing the second compound according to the present invention is a domain selected from the group consisting of a cysteine, histidine-dependent amidohydrolases/peptidase (CHAP) domain, an endopeptidase domain, and an amidase domain; all preferably as described previously herein.

Preferably, a first, second, third and/or further polypeptide together encompassing the second compound according to the present invention comprises a different multiplicity of a first, second, third and/or further enzymatic active domain according to the present invention. A “multiplicity” is herein defined as a number of copies. A “different multiplicity” is defined herein as a multiplicity or number of copies of a specific enzymatic active domain according to the invention, i.e. a first, second, third or further enzymatic active domain as defined herein, comprised within a specific polypeptide of the invention, i.e. a first, second, third or further polypeptide as defined herein, to be different form a multiplicity or number of copies of that same enzymatic active domain within another polypeptide of the combination encompassing the second compound of the invention. For example, a combination encompassing the second compound of the present invention comprises a first polypeptide comprising a specific number of copies of a first enzymatic active domain, and a second polypeptide comprising a different number of copies of said first enzymatic active domain. Furthermore, said first polypeptide of said exemplified combination encompassing the second compound of the present invention may further comprise a specific number of copies of second enzymatic active domain, which is different from the number of copies of said second enzymatic active domain as comprised on said second polypeptide of said combination. Furthermore, any further polypeptide of said exemplified combination encompassing the second compound of the present invention may comprise a number of copies of further enzymatic active domain, which is different from the number of copies of said further enzymatic active domain as comprised on said first and second polypeptide of said combination. Although a combination of distinct polypeptides each comprising a single distinct enzymatic active domain showed synergistic lytic activity as compared to the lytic activity of each separate polypeptide, it was surprisingly found by the present inventors that polypeptides comprising a multiplicity of enzymatic active domains show superior lytic activity as compared to polypeptides comprising a single enzymatic active domain.

Moreover, a combination of distinct enzymatic domains on distinct polypeptides wherein at least one of said distinct polypeptides comprises a multiplicity of enzymatic active domains was found superior over a combination wherein all said distinct polypeptides comprise a single distinct enzymatic active domain. Moreover, a combination encompassing the second compound according to the present invention, wherein a first, second, third and/or further polypeptide comprise a multiplicity of a first, second, third and/or further enzymatic active domain according to the present invention, respectively, was found superior over a combination encompassing the second compound according to the present invention, wherein said first, second, third and/or further polypeptide comprise a single copy of said first, second, third and/or further enzymatic active domain, respectively, and preferably wherein said multiplicity, as defined herein, is 2, i.e. a duplicate. In a preferred embodiment, the synergistic effect of a combination encompassing the second compound according to the present invention, wherein a first, second, third and/or further polypeptide according to the present invention comprise a multiplicity of a first, second, third and/or further enzymatic active domain according to the present invention, respectively, was found superior over a combination encompassing the second compound according to the present invention, wherein said first, second, third and further polypeptide comprise a single copy of said first, second, third and further enzymatic active domain, respectively, and preferably wherein said multiplicity, as defined herein below, is 2, i.e. a duplicate.

Preferably, a first and/or second polypeptide of a combination encompassing the second compound according to the present invention, comprises a different multiplicity of a first and/or second enzymatic active domain according to the present invention. Multiplicity of said first and second domain is defined as previously herein as a number of copies, preferably indicated by k, l, n and p, of said first and second domain indicated as follows:
k indicates the number of copies of said first enzymatic active domain on said first polypeptide;
l indicates the number of copies of said second enzymatic active domain on said first polypeptide;
n indicates the number of copies of said first enzymatic active domain on said second polypeptide;
p indicates the number of copies of said second enzymatic active domain on said second polypeptide;
   and wherein k and p are independent integers from 1-10, 1-9, 1-8, 1-7, 1-6, 1-5, 1-4, 1-3, or preferably 1-2, and l and n are independent integers from 0-10, 0-9, 0-8, 0-7, 0-6, 0-5, 0-4, 0-3, or preferably 0-2, and wherein k is a different integer than n and/or l is a different integer than p, most preferably k and p are 2 and l and n are 0.

Preferably, a first, second and third polypeptide encompassing the second compound of the present invention comprise a different multiplicity of a first, second and third enzymatic active domain according to the present invention.

Multiplicity of said first, second and third domain is defined as previously herein as a number of copies, preferably indicated by k, l, m, n, p, q, r, s and t, of said first, second and third domain indicated as follows:
k indicates the number of copies of said first enzymatic active domain on said first polypeptide;
l indicates the number of copies of said second enzymatic active domain on said first polypeptide;
m indicates the number of copies of said third enzymatic active domain on said first polypeptide;
n indicates the number of copies of said first enzymatic active domain on said second polypeptide;
p indicates the number of copies of said second enzymatic active domain on said second polypeptide;
q indicates the number of copies of said third enzymatic active domain on said second polypeptide;
r indicates the number of copies of said first enzymatic active domain on said third polypeptide;
s indicates the number of copies of said second enzymatic active domain on said third polypeptide;
t indicates the number of copies of said third enzymatic active domain on said third polypeptide;
   and wherein k, p and t are independent integers from 1-10, 1-9, 1-8, 1-7, 1-6, 1-5, 1-4, 1-3, or preferably 1-2, and l, m, n, q, r, and s are independent integers from 0-10, 0-9, 0-8, 0-7, 0-6, 0-5, 0-4, 0-3, or preferably 0-2, and wherein k is a different integer than n and/or r, and/or l is a different integer than p and/or s, and/or t is a different integer than m or q, most preferably k, p and t are 2 and l, m, n, q, r, and s are 0.

Preferably, a first, second, third and further polypeptide encompassing the second compound of the present invention comprise a different multiplicity of a first, second, third and further enzymatic active domain according to the present invention. Multiplicity of said further enzymatic active domain in view of said first, second and third enzymatic active domain is to be construed herein in an analogous manner as defined herein above for a first, second and third enzymatic active domain.

Preferably a first, second, third or further polypeptide encompassing the second compound according to the present invention has a length of at least 140, 150, 160, 170, 180, 190, 200, 210, 220, 230, 240, 250, 260, 270, 280, 290, 300, 310, 320 or 330 amino acids and/or a length of at most 850, 800, 750, 700, 650, 600, 550, 500, 490, 480, 470, 460, 450, 440, 430, 420, 410, 400, 390, 380 or 370 amino acids. More preferably, a first, second or third polypeptide encompassing the second compound according to the present invention has a length of 140-850, 140-800, 140-750, 140-700, 140-650, 140-600, 140-550 140-500, 140-490, 140-480, 140-470, 140-460, 140-450, 140-440, 140-430, 140-420, 140-410, 140-400, 140-390, 140-380, 140-370, 150-850, 160-850, 170-850, 180-850, 190-850, 200-850, 210-850, 220-850, 230-850, 240-850, 250-850, 260-850, 270-850, 280-850, 290-850, 300-850, 310-850, 320-850 or 330-850 amino acids.

Preferably a first and second polypeptide encompassing the second compound according to the present invention each have a length of at least 140, 150, 160, 170, 180, 190, 200, 210, 220, 230, 240, 250, 260, 270, 280, 290, 300, 310, 320 or 330 amino acids and/or a length of at most 800, 850, 700, 650, 600, 550, 500, 490, 480, 470, 460, 450, 440, 430, 420, 410, 400, 390, 380 or 370 amino acids. More preferably, a first and second polypeptide according to the present invention each have a length of 140-850, 140-800, 140-750, 140-700, 140-650, 140-600, 140-550 140-500, 140-490, 140-480, 140-470, 140-460, 140-450, 140-440, 140-430, 140-420, 140-410, 140-400, 140-390, 140-380, 140-370, 150-850, 160-850, 170-850, 180-850, 190-850, 200-850, 210-850, 220-850, 230-850, 240-850, 250-850, 260-850, 270-850, 280-850, 290-850, 300-850, 310-850, 320-850 or 330-850 amino acids.

Preferably a first, second and third polypeptide encompassing the second compound according to the present invention each have a length of at least 140, 150, 160, 170, 180, 190, 200, 210, 220, 230, 240, 250, 260, 270, 280, 290, 300, 310, 320 or 330 amino acids and/or a length of at most 800, 850, 700, 650, 600, 550, 500, 490, 480, 470, 460, 450, 440, 430, 420, 410, 400, 390, 380 or 370 amino acids. More preferably, a first, second and third polypeptides encompassing the second compound according to the present invention each have a length of 140-850, 140-800, 140-750, 140-700, 140-650, 140-600, 140-550 140-500, 140-490, 140-480, 140-470, 140-460, 140-450, 140-440, 140-430, 140-420, 140-410, 140-400, 140-390, 140-380, 140-370, 150-850, 160-850, 170-850, 180-850, 190-850, 200-850, 210-850, 220-850, 230-850, 240-850, 250-850, 260-850, 270-850, 280-850, 290-850, 300-850, 310-850, 320-850 or 330-850 amino acids.

Preferably a first, second, third and further polypeptide encompassing the second compound according to the present invention each have a length of at least 140, 150, 160, 170, 180, 190, 200, 210, 220, 230, 240, 250, 260, 270, 280, 290, 300, 310, 320 or 330 amino acids and/or a length of at most 800, 850, 700, 650, 600, 550, 500, 490, 480, 470, 460, 450, 440, 430, 420, 410, 400, 390, 380 or 370 amino acids. More preferably, a first, second, third and further polypeptides encompassing the second compound according to the present invention each have a length of 140-850, 140-800, 140-750, 140-700, 140-650, 140-600, 140-550 140-500, 140-490, 140-480, 140-470, 140-460, 140-450, 140-440, 140-430, 140-420, 140-410, 140-400, 140-390, 140-380, 140-370, 150-850, 160-850, 170-850, 180-850, 190-850, 200-850, 210-850, 220-850, 230-850, 240-850, 250-850, 260-850, 270-850, 280-850, 290-850, 300-850, 310-850, 320-850 or 330-850 amino acids.

An embodiment provides a combination of a source of a first and a second enzymatic active domain encompassing the second compound according to the present invention, wherein said first and second enzymatic active domains are comprised on distinct, first and second polypeptides of the present invention, wherein said first polypeptide is free of said second enzymatic active domain and said second polypeptide is free of said first enzymatic active domain. Moreover, provided is a combination according to the present invention, wherein l and n are 0.

Another embodiment provides a combination of a source of a first, second and third enzymatic active domain encompassing the second compound according to the present invention, wherein said first, second and third enzymatic active domains are comprised on distinct, first, second and third polypeptides, wherein said first polypeptide is free of said second and third enzymatic active domain, said second polypeptide is free of said first and third enzymatic active domain, and said third polypeptide is free of said first and second enzymatic active domain. Moreover, provided is a combination according to the present invention, wherein l, m, n, q, r and s are 0. Even more preferably, the present invention provides a combination encompassing the second compound according to the present invention, wherein l, m, n, q, r and s are 0 and k, p and t are 2.

Another embodiment provides a combination of a source of a first, second, third and further enzymatic active domain encompassing the second compound according to the present invention, wherein said first, second, third and further enzymatic active domains are comprised on a distinct, first, second, third and further polypeptide, respectively, wherein

preferably said first polypeptide is free of said second, third and further enzymatic active domain;

preferably said second polypeptide is free of said first, third and further enzymatic active domain;

preferably said third polypeptide is free of said first, second and further enzymatic active domain; and,

preferably said further polypeptide is free of said first, second and third enzymatic active domain.

Preferably said first, second, third and further enzymatic active domain are comprised within said first, second, third and further polypeptide, respectively, in duplicate, i.e. wherein the multiplicity as identified herein is 2. Also encompassed is a combination encompassing the second compound according to the present invention, wherein a first, second and/or third polypeptide according to the present invention are not free of a first, second and/or third enzymatic active domain according to the present invention, but said first, second and/or third polypeptide differ in multiplicity of said first, second and/or third enzymatic active domain. Moreover, encompassed is a combination encompassing the second compound according to the present invention, wherein at least one of k, l, m, n p, q, r, s or t is 2 and wherein any of the other k, l, m, n p, q, r, s and/or t is 1 or 0.

Preferred is a combination encompassing the second compound according to the present invention, wherein a first, second, third and/or further polypeptide is a polypeptide that has at least 80, 81, 82, 83, 84, 85, 86, 87, 88, 89, 90, 91, 92, 93, 94, 95, 96, 97, 98, 99 or 100% identity with a polypeptide selected from the group consisting of SEQ ID NO: 26, 28, 30, 32, 34, 36, 38, 40, 42, 44, 46, 48, 50, 52, 54, 56, 58, 60, 62, 64, 66, 68, 70, 72, 76, 78, 80, 82, 84, 86, 88, 90, 92, 94, 98 or 100.

Within the context of the present invention, several preferred, non-limiting, combinations encompassing the second compound according to the invention are envisaged, which are listed here below.

Preferred is a combination of a source of first enzymatic active domain and a second enzymatic active domain, wherein said first and second enzymatic active domains are comprised on distinct first and second polypeptides, and wherein said first enzymatic active domain is a cysteine, histidine-dependent amidohydrolases/peptidase domain and said second enzymatic active domain is an endopeptidase domain or wherein said first enzymatic active domain is a cysteine, histidine-dependent amidohydrolases/peptidase domain and said second enzymatic active domain is amidase domain or wherein said first enzymatic active domain is an endopeptidase domain and said second enzymatic active domain is amidase domain, wherein said distinct first and second each further comprises a cell wall-binding domain, and wherein each of said distinct first and second polypeptides comprises a multiplicity of said first or second enzymatic active domain, preferably said multiplicity being 2, i.e. a duplicate.

Also preferred is a combination of a source of first and second enzymatic active domain, wherein said first and second enzymatic active domains are comprised on distinct first and second polypeptides, and wherein said first enzymatic domain is histidine-dependent amidohydrolases/peptidase domain and said second enzymatic active domain is an endopeptidase domain or said first enzymatic active domain is a cysteine, histidine-dependent amidohydrolases/peptidase domain and said second enzymatic active domain is amidase domain or said first enzymatic active domain is an endopeptidase domain and said second enzymatic active domain is amidase domain, and wherein said first and second polypeptide each further comprise a cell wall binding domain.

Also preferred is a combination of a source of first enzymatic active domain and a second enzymatic active domain, wherein said first and second enzymatic active domains are comprised on distinct first and second polypeptides, and wherein said first enzymatic active domain is a cysteine, histidine-dependent amidohydrolases/peptidase domain and said second enzymatic active domain is an endopeptidase domain, and wherein said combination further comprises a source of a third enzymatic active domain comprised on a distinct third polypeptide, wherein said third enzymatic active domain is an amidase domain and said distinct first, second and third polypeptide each further comprises a cell wall-binding domain, and wherein each of said distinct first, second and third polypeptides comprises a multiplicity of said first, second or third enzymatic active domain, preferably said multiplicity being 2, i.e. a duplicate.

Also preferred is a combination of a source of first, second and third enzymatic active domain, wherein said first, second and third enzymatic active domains are comprised on distinct first, second and third polypeptides, and wherein said first enzymatic domain is histidine-dependent amidohydrolases/peptidase domain, said second enzymatic active domain is an endopeptidase domain and said third enzymatic active domain is an amidase domain, and wherein said first, second and third polypeptide each further comprise a cell wall binding domain.

Also preferred is a combination wherein, a first enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 10 and a second enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 16.

Also preferred is a combination wherein, a first enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 10 and a second enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 18.

Also preferred is a combination wherein, a first enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 16 and a second enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 18.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

More preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34.

More preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination, wherein a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52 and a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46.

Also preferred is a combination wherein, a first enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 10, a second enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 16 and a third enzymatic active domain according to the present invention has at least 80, 81, 82, 83, 84, 85, 86, 87, 88, 89, 90, 91, 92, 93, 94, 95, 96, 97, 98, 99 or 100% identity with SEQ ID NO: 18.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 32, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 44 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 26.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 26.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 36, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 48 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 30.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 32, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 26.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 44 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 26.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 32, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 44 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 32, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 44 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 50.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 56, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 68 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 50.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 60, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 72 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 54.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 56, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 50.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 68 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 50.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 56, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 68 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 65, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 68 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 52.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 58, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 46 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

Also preferred is a combination wherein, a first polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 34, a second polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 70 and a third polypeptide according to the present invention has at least 80, 81, 82, 83, 84, 85, 86, 87, 88, 89, 90, 91, 92, 93, 94, 95, 96, 97, 98, 99 or 100% identity with SEQ ID NO: 28.

It is to be understood that a combination as described herein encompassing the second compound according to the present invention includes mixtures of a source of a first, a source of a second and optionally a source of a third and/or further enzymatic active domain according to in varying ratios. Preferably, such combination comprises a source a first and a source a second enzymatic active domain according to the present invention, wherein said first and second enzymatic active domain are present in equimolar amounts. Also preferred is a combination comprising a source a first, a source a second and a source a third enzymatic active domain according to the present invention, wherein said first, second and third enzymatic active domain are present in equimolar amounts. Also preferred is a combination comprising a source of a first, a source of a second, a source of a third and a source of a further enzymatic active domain according to the present invention, wherein said first, second, third and further enzymatic active domain are present in equimolar amounts.

In a second aspect, the invention provides for a kit of parts comprising:
a) a first vial containing a first composition comprising a first compound as defined in the first aspect of the invention; and,
b) a second vial containing a second composition comprising a second compound as defined in the first aspect of the invention; and optionally,
c) instructions for use, preferably comprising a dosage regime.
   Preferably, said kit of parts, more specifically said first and second composition of said kit of parts, is for use as a medicament, preferably for use in the treatment of eczema, more preferably for use in the treatment of atopic dermatitis, as further detailed herein. A dosage regime is to be understood herein as an instruction for administration to an individual in the need thereof, preferably an instruction indicating an administration route, administration frequency and administration dosage, and optionally an instruction for admixing said first and second compound just before administration, as required for treatment, preferably required for treatment of eczema, more preferably for treatment of atopic dermatitis. Preferred administration routes, frequencies and dosages are further detailed herein. In an embodiment, said first composition according to a second aspect and/or said second composition according to a second aspect of the present invention is administered separately, preferably as part of an overall treatment regimen. In an alternative embodiment, said first composition according to a second aspect and said second composition according to a second aspect of the present invention are stored separately, and admixed just before administration. Preferably, “just before” is to be understood herein as less than 120, 60, 30, 15, 5, 4, 3, 2 or 1 minutes before administration, preferably less than 5 minutes before administration.

Said first and said second vial may be any vial, bottle, tube, ampoule, container, flask or the like, suitable for storing said first and second composition as defined herein, respectively. Preferably said first and/or second vial has a volume of between 0.1 and 500 mL, preferably between 1 and 100 mL, more preferably of about 5, 10, 50 or 100 mL.

In a third aspect, the invention provides for a method of treatment comprising the administration of a composition according to the first aspect of the invention and/or the sequential or simultaneous administration of a first and second compound of a kit of parts according to the second aspect of the invention.

Preferably, said method of treatment is a method for preventing, delaying and/or curing an infectious disease, such as, but not limited to, a skin infection, mastitis, pneumonia, meningitis, endocarditis, Toxic Shock Syndrome (TSS), sepsis, septicemia, bacteremia, or osteomyelitis. Preferably, said skin infection is selected from the group of acne, rosacea, pimples, impetigo, boils, furuncles, cellulitis folliculitis, psoriasis, carbuncles, scaled skin syndrome and abscesses. Preferably, said method of treatment is a method for preventing, delaying and/or curing eczema such as atopic dermatitis, allergic contact eczema, contact eczema, dyshidrotic eczema, neurodermatitis, nummular eczema, seborrheic eczema, stasis dermatitis, preferably atopic dermatitis. Preferably, said method of treatment is topical treatment of a skin infection and/or eczema as identified herein, more preferably atopic dermatitis.

Encompassed in the present invention is a composition according to the first aspect of the invention and/or a kit of parts according to second aspect of the invention, for use as a medicament. Preferably, said composition according to the first aspect of the invention and/or said kit of parts according to second aspect of the invention is for preventing, delaying and/or curing an infectious disease, such as, but not limited to, a skin infection, mastitis, pneumonia, meningitis, endocarditis, Toxic Shock Syndrome (TSS), sepsis, septicemia, bacteremia, or osteomyelitis. Preferably, said skin infection is selected from the group of acne, rosacea, pimples, impetigo, boils, furuncles, cellulitis, folliculitis, psoriasis, carbuncles, scaled skin syndrome and abscesses. Preferably, said composition according to the first aspect of the invention and/or said kit of parts according to the second aspect of the invention is for use preventing, delaying and/or curing of eczema such as atopic dermatitis, allergic contact eczema, contact exzema, dyshidrotic eczema, neurodermatitis, nummular eczema, seborrheic eczema, stasis dermatitis, preferably atopic dermatitis.

Also encompassed in the present invention is the use of a composition according to the first aspect of the invention and/or a kit of parts according to second aspect of the invention for the manufacture of a medicament. Preferably, said medicament is for preventing, delaying and/or curing an infectious disease, such as, but not limited to, a skin infection, mastitis, pneumonia, meningitis, endocarditis, Toxic Shock Syndrome (TSS), sepsis, septicemia, bacteremia, or osteomyelitis. Preferably, said skin infection is selected from the group of acne, rosacea, pimples, impetigo, boils, furuncles, cellulitis, folliculitis, psoriasis, carbuncles, scaled skin syndrome and abscesses. Preferably, said medicament is a medicament for preventing, delaying and/or curing eczema such as atopic dermatitis, allergic contact eczema, contact eczema, dyshidrotic eczema, neurodermatitis, nummular eczema, seborrheic eczema, stasis dermatitis, preferably atopic dermatitis.

Preferably, said composition according to the first aspect, a first composition of a kit of parts according to the second aspect and/or a second composition of a kit of parts according to the second aspect and/or medicament as defined herein is a topical formulation understood herein as a formulation, including a microencapsulated formulation, being suitable for topical administration and may be in the form of a cream, ointment, solution, powder, spray, aerosol, capsule, solid or gel, and/or may be bonded to a solid surface, e.g. by immobilization with affinity ligands or through ionic/hydrophobic interactions and covalent immobilization.

A composition according to the first aspect of the invention and/or a first and/or second composition of a kit of parts according to the second aspect of the invention may also form part of a body wash, soap, application stick or cosmetic.

A composition according to the first aspect and/or a second composition of a kit of parts according to the second aspect and/or a mixture resulting from admixing said first and second composition of a kit of parts according to the second aspect just before administration as earlier indicated herein, is preferably said to be active, functional or therapeutically active when it decreases the amount of bacterial cells, preferably gram positive bacterial cells, more preferably the amount of Staphylococcus bacterial cells, most preferably the amount of Staphylococcus aureus bacterial cells, present in a patient or in a cell of said patient or in a cell line or in a cell free in vitro system and preferably means that 99%, 90%, 80%, 70%, 60%, 50%, 40%, 30%, 20%, 10%, 5% or less of the initial amount of said bacterial cells is still detectable. More preferably, no bacterial cell, preferably no gram positive bacterial cell, more preferably no Staphylococcus bacterial cell, most preferably no Staphylococcus aureus bacterial cell, is detectable. In this paragraph, the expression “amount of bacterial cells” preferably means viable bacterial cells. Staphylococci of all genera may be detected using standard techniques known by the artisan such as immunohistochemical techniques using Staphylococcus specific antibodies, tube coagulase tests that detect staphylocoagulase or “free coagulase”, detection of surface proteins such as clumping factor (slide coagulase test) and/or protein A (commercial latex tests). Viable Staphylococci may be detected using standard techniques known by the artisan such as microbiological bacterial culture techniques and/or real-time quantitative reverse transcription polymerase chain reaction to assay for bacterial mRNA. A decrease in amount of bacterial cells according to the present invention is preferably assessed in a tissue or in a cell of an individual or a patient by comparison to the amount present in said individual or patient before treatment with said composition or polypeptide of the invention. Alternatively, the comparison can be made with a tissue or cell of said individual or patient which has not yet been treated with said composition or polypeptide in case the treatment is local.

Preferably a composition according to the first aspect of the invention and/or a second composition of a kit of parts of the second aspect of the invention, and/or a resulting mixture resulting from admixing the first and second composition of the kit of part of the second aspect of the invention just before administration as identified herein before comprises an amount of a second compound as defined herein which is therapeutically active as earlier identified herein. Preferably, said composition is for topic administration to an individual in the need thereof, preferably to a patient suffering from eczema, and comprises said second compound in an effective amount, preferably a concentration of 0.001-10% by weight of the total composition. Depending on the specific activity of the second compound, the effective amount may be as low about a few micrograms/ml such as about 1, 2, 3, 4, 5, 6, 7, 8, 9, 10 microgram/ml to about several milligrams/ml such as about 1, 2, 3, 4, 5, 6, 7, 8, 9, 10 milligram/ml

Preferably a composition according to the first aspect of the invention and/or a first composition of a kit of parts of the second aspect of the invention, and/or a resulting mixture resulting from admixing the first and second composition of the kit of part of the second aspect of the invention just before administration as identified herein before is a composition for topic application for the treatment of eczema comprising an anti-inflammatory compound, selected from, but not limited to, a corticosteroid a calcineurin inhibitor, an immunotherapeutic compound, a recombinant human IFN-gamma, a microbial probiotic, a cytokine modulator, an inflammatory cell recruitment blocker, a T cell activation inhibitor, or a combination of these. Preferably, said composition is for topic administration to an individual in the need thereof, preferably to a patient suffering from eczema, and comprises said anti-inflammatory compound compound in an effective amount, preferably in the range of 0.01 to 10% by weight of the total composition, preferably in the range of 0.05 to 5%, more preferably in a range of 0.05 to 2.5%, even more preferably in a range of 0.1 to 1%.

Preferably a composition according to the first aspect of the invention and/or a first composition of a kit of parts of the second aspect of the invention, and/or a resulting mixture resulting from admixing the first and second composition of the kit of part of the second aspect of the invention just before administration as identified herein before is a composition for topic application for the treatment of eczema comprising a corticosteroid in the range of 0.05 to 5%, by weight of the total composition preferably in a range of 0.05 to 2.5% more preferably in a range of 0.1 to 1%, even more preferably comprising about 0.05% clobetasol propionate, about 0.05% halobetasol propionate, about 0.1% fluocinonide, about 0.05% diflorasone diacetate, about 0.1% mometasone furoate, about 0.1% halcinonide, about 0.25% desoximetasone, about 0.05% fluocinonide, about 0.05% desoximetasone, about 0.1% clocortolone pivalate, about 0.1% mometasone furoate, about 0.1% triamcinolone acetonide, about 0.1% betamethasone valerate, about 0.025% fluocinolone acetonide, about 0.05% fluticasone propionate, about 0.1% prednicarbate, about 0.1% hydrocortisone probutate, about 0.1% triamcinolone acetonide, about 0.05% alclometasone dipropionate, about 0.05% desonide, about 0.025% triamcinolone acetonide, about 0.1% hydrocortisone butyrate, about 0.01% fluocinolone acetonide, about 1% hydrocortisone acetate, about 2% hydrocortisone, about 2.5% hydrocortisone and/or 0.5-1% hydrocortisone. About is defined herein as a value minus or plus 10% of the indicated value.

A composition according to the first aspect, a first composition of a kit of parts according to the second aspect and/or a second composition of a kit of parts according to the second aspect and/or medicament as defined herein may be in the liquid, solid or semi-liquid or semi-solid form, preferably further comprising a pharmaceutical acceptable carrier, excipient and/or stabilizer. Examples of pharmaceutically acceptable carriers, excipients and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose mannose, or dextrins; chelating agents such as EDTA; sugar alcohols such as mannitol or sorbitol; salt-forming counterions such as sodium; and/or nonionic surfactants such as polysorbate (TWEEN™), polyethylene glycol (PEG), and poloxamers (PLURONICS™); and polymer thickeners such as hydrophilic and hydroalcoholic gelling agents frequently used in the cosmetic and pharmaceutical industries, preferably a gelling agent comprises between about 0.2% and about 4% by weight composition. A composition according to the first aspect, a first composition of a kit of parts according to the second aspect and/or a second composition of a kit of parts according to the second aspect and/or medicament as defined herein can also include a lubricant, a wetting agent, a sweetener, a flavoring agent, an emulsifier, a suspending agent, a sun screen such as, but not limited to, titanium dioxide or methyl cinnamate, and/or a preservative, in addition to the above ingredients, preferably a preservative is present as about 0.05% to 0.5% by weight of the total composition. A composition according to the first aspect, a first composition of a kit of parts according to the second aspect and/or a second composition of a kit of parts according to the second aspect and/or medicament as defined herein can also include a carrier which are known in the art (such as a carbohydrate and a sugar-alcohol) to aid in the exposure of the skin to a medicament.

Preferably, a composition according to the first aspect, a first composition of a kit of parts according to the second aspect and/or a second composition of a kit of parts according to the second aspect and/or a medicament as defined herein further comprises and additional active ingredient. An additional active ingredient may be any of, but is not limited to, an anti-inflammatory agent; a standard or conventional antibiotic agent such as, but not limited to penicillin, synthetic penicillins, bacitracin, methicillin, cephalosporin, polymyxin, cefaclor, Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef mafate and/or chelating agents; an antifungal, such as, but not limited to, oxiconazole nitrate, ciclopirox olamine, ketoconazole, miconazole nitrate and butoconazole nitrate; an anti-androgen, such as, but not limited to, flutamide and/or finasterid; a local anesthetic agent, such as, but not limited to tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate and/or pramoxine hydrochloride; and dapsone which has both antimicrobial and anti-inflammatory properties. Preferred weight percentages of antimicrobial agents are 0.1% to 10% weight of the total composition. Preferred weight percentages for local anesthetics are 0.025% to 5% by weight of the total composition.

A composition according to the first aspect, a first composition according to a second aspect and/or a second composition according to a second aspect and/or medicament as defined herein can be used to treat animals, including humans, suffering from any of the an infectious diseases and/or eczema as identified herein above, preferably from atopic dermatitis.

A preferred route of administration of said composition and/or said medicament is any suitable route of administration that can be used to administer said composition according to the first aspect, said first composition according to a second aspect and/or said second composition according to a second aspect and/or medicament as defined herein including but not limited to: oral, aerosol or other device for delivery to the lungs, nasal spray, intravenous, intramuscular, intraperitoneal, intrathecal, vaginal, rectal, topical, lumbar puncture, intrathecal, and direct application to the brain and/or meninges. Preferably, said composition according to the first aspect, said first composition according to a second aspect and/or said second composition according to a second aspect and/or medicament as defined herein are administered topical, preferably at the side of infection and/or lesion and/or, in the case of eczema, preferably at the side of skin rash.

A preferred administration frequency of said composition and/or said medicament is once or twice a day, preferably to the area of the skin affected by the disease or the side of lesion known in the art as the side of rash. Preferably said treatment is continued as long as required for the rash to be cleared. Preferably said treatment is continued for 2 to 3 days, for 7 to 10 days and/or for 2 to 3 weeks. Preferably a total amount of composition for topic application is administered as identified herein resulting in a total application of about 1 gram of corticosteroid to the person in the need thereof.

A preferred dosage of administration of said composition and/or said medicament is a dosage containing an effective total amount of said first and second compound resulting in the prevention, delay and/or cure of an infectious disease and/or eczema as earlier identified herein, preferably eczema, more preferably atopic dermatitis.

### DEFINITIONS

“Sequence identity” or “identity” in the context of amino acid- or nucleic acid-sequence is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. Within the present invention, sequence identity with a particular sequence preferably means sequence identity over the entire length of said particular polypeptide or polynucleotide sequence. The sequence information as provided herein should not be so narrowly construed as to require inclusion of erroneously identified bases. The skilled person is capable of identifying such erroneously identified bases and knows how to correct for such errors.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A polynucleotide is represented by a nucleotide sequence. A polypeptide is represented by an amino acid sequence. A nucleic acid construct is defined as a polynucleotide which is isolated from a naturally occurring gene or which has been modified to contain segments of polynucleotides which are combined or juxtaposed in a manner which would not otherwise exist in nature. Optionally, a polynucleotide present in a nucleic acid construct is operably linked to one or more control sequences, which direct the production or expression of said peptide or polypeptide in a cell or in a subject.

As used herein the term “heterologous sequence” or “heterologous nucleic acid” is one that is not naturally found operably linked as neighboring sequence of said first nucleotide sequence. As used herein, the term “heterologous” may mean “recombinant”. “Recombinant” refers to a genetic entity distinct from that generally found in nature. As applied to a nucleotide sequence or nucleic acid molecule, this means that said nucleotide sequence or nucleic acid molecule is the product of various combinations of cloning, restriction and/or ligation steps, and other procedures that result in the production of a construct that is distinct from a sequence or molecule found in nature.

“Operably linked” is defined herein as a configuration in which a control sequence is appropriately placed at a position relative to the nucleotide sequence coding for the polypeptide of the invention such that the control sequence directs the production/expression of the peptide or polypeptide of the invention in a cell and/or in a subject.

“Operably linked” may also be used for defining a configuration in which a sequence is appropriately placed at a position relative to another sequence coding for a functional domain such that a chimeric polypeptide is encoded in a cell and/or in a subject.

Expression will be understood to include any step involved in the production of the peptide or polypeptide including, but not limited to, transcription, post-transcriptional modification, translation, post-translational modification and secretion.

Optionally, a promoter represented by a nucleotide sequence present in a nucleic acid construct is operably linked to another nucleotide sequence encoding a peptide or polypeptide as identified herein.

The term “transformation” refers to a permanent or transient genetic change induced in a cell following the incorporation of new DNA (i.e. DNA exogenous to the cell). When the cell is a bacterial cell, as is intended in the current invention, the term usually refers to an extrachromosomal, self-replicating vector which harbors a selectable antibiotic resistance.

An expression vector may be any vector which can be conveniently subjected to recombinant DNA procedures and can bring about the expression of a nucleotide sequence encoding a polypeptide of the invention in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context of the invention, a promoter preferably ends at nucleotide -1 of the transcription start site (TSS).

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The term “control sequences” is defined herein to include all components, which are necessary or advantageous for the expression of a polypeptide. Each control sequence may be native or foreign to the nucleic acid sequence encoding the polypeptide. Such control sequences include, but are not limited to, a leader, optimal translation initiation sequences (as described in Kozak, 1991, J. Biol. Chem. 266:19867-19870), a polyadenylation sequence, a pro-peptide sequence, a pre-pro-peptide sequence, a promoter, a signal sequence, and a transcription terminator. At a minimum, the control sequences include a promoter, and transcriptional and translational stop signals.

The control sequences may be provided with linkers for the purpose of introducing specific restriction sites facilitating ligation of the control sequences with the coding region of the nucleic acid sequence encoding a polypeptide.

The control sequence may be an appropriate promoter sequence, a nucleic acid sequence, which is recognized by a host cell for expression of the nucleic acid sequence. The promoter sequence contains transcriptional control sequences, which mediate the expression of the polypeptide. The promoter may be any nucleic acid sequence, which shows transcriptional activity in the cell including mutant, truncated, and hybrid promoters, and may be obtained from genes encoding extracellular or intracellular polypeptides either homologous or heterologous to the cell.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator, which is functional in the cell, may be used in the present invention.

The control sequence may also be a suitable leader sequence, a non-translated region of a mRNA which is important for translation by the host cell. The leader sequence is operably linked to the 5′ terminus of the nucleic acid sequence encoding the polypeptide. Any leader sequence, which is functional in the cell, may be used in the present invention.

The control sequence may also be a polyadenylation sequence, a sequence which is operably linked to the 3′ terminus of the nucleic acid sequence and which, when transcribed, is recognized by the host cell as a signal to add polyadenosine residues to transcribed mRNA. Any polyadenylation sequence, which is functional in the cell, may be used in the present invention.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### EXAMPLES

Use of Corticosteroids and a Compound Specifically Targeting S. aureus (Staphefekt™)

In several forms of dermatitis, courses of topical corticosteroid therapy are used to suppress symptoms of local inflammation. However, corticosteroids do not treat the underlying cause of the inflammation, and symptoms are known to return eventually. It was hypothesized that a second compound according to the present invention would be effective in combination with corticosteroid treatment. As a second compound according to the invention, a compound specifically targeting S. aureus was used. The compound Staphefekt™ (Gladskin™) was obtained from Micreos Human Health B.V., The Netherlands. Since Staphefekt™ eradicates S. aureus as an etiological factor of local inflammation, it was speculated that during Staphefekt™ treatment less corticosteroids would be needed to alleviate symptoms.

To study the use of corticosteroid during Staphefekt™ treatment, corticosteroid use and symptom relief was monitored in eight patients, of whom the treatment of several kinds of dermatitis with Staphefekt™ was guided and observed by a physician (table 1). In the six cases where S. aureus was found, symptoms diminished during treatment with Staphefekt™, and patients reported less need of corticosteroids anamnestically. In one patient with severe constitutional eczema, the burden of S. aureus carriership was high and symptoms diminished only moderately with Staphefek™, necessitating the unchanged use of corticosteroids and eventually even immune suppression with Neoral. In one case where no S. aureus was found, Staphefekt™ had no effect on symptoms and corticosteroid use remained unchanged.

The use of corticosteroids was not completely abandoned in the successfully treated patients, especially with the recurrence of symptoms after ceasing Staphefekt™ use. This suggests that a quick relief of symptoms at the stage of local inflammation is best achieved by combining symptomatic corticosteroid therapy with eradication of etiological S. aureus carriership by Staphefekt™.

The reduced need for corticosteroids as observed in customer feedback, questionnaires and the present physician guided Staphefekt™ study clearly indicates that a lower dose or shorter course of corticosteroids would be effective in combination therapy with a second compound according to the present invention, in casu Staphefekt™.

TABLE 1
Physician guided Staphefekt ™ cases.

| | | | | | corticosteroid |
|---|---|---|---|---|---|
| gender | treatment | | S. aureus | relief of | use during |
| (age) | indication | site | carriership | symptoms | treatment |
| male | eczema | neck/face | +++ | moderate | unchanged |
| (22) | | | | | corticosteroid |
| | | | | | use, Neoral |
| | | | | | because of |
| | | | | | relapse |
| male | eczema | face | − | yes | less need |
| (32) | | | | | |
| female | eczema | arm, knee | + | yes | no need |
| (16) | | | | | |
| female | peri-oral | peri-oral | + | yes | less need |
| (19) | dermatitis | | | | until relapse |
| female | eczema | arm | − | no | unchanged |
| (16) | | | | | |
| male | contact | hands | +++ | yes | less need |
| (28) | dermatitis | | | | |
| male | contact | hands | + | yes | no need |
| (57) | dermatitis | | | | |
| female | eczema | arm | + | yes | less need |
| (30) | | | | | |

TABLE 2
SEQ ID NO overview table

| SEQ ID NO | Name construct | organism |
|---|---|---|
| 1 | Ply2638 endolysin CDS | Bacteriophage 2638A |
| 2 | Ply2638 endolysin PRT | Bacteriophage 2638A |
| 3 | CWT-LST CDS | S. simulans |
| 4 | CWT-LST PRT | S. simulans |
| 5 | CBD2638 CDS | Bacteriophage 2638A |
| 6 | CBD2638 PRT | Bacteriophage 2638A |
| 7 | CWT-NM3 CDS | S. aureus phage phiNM3 |
| 8 | CWT-NM3 PRT | S. aureus phage phiNM3 |
| 9 | CHAPK CDS | S. phage K |
| 10 | CHAPK PRT | S. phage K |
| 11 | CHAP-ΦTwort CDS | S. phage Twort |
| 12 | CHAP-ΦTwort PRT | S. phage Twort |
| 13 | M23-2638 CDS | Bacteriophage 2638A |
| 14 | M23-2638 PRT | Bacteriophage 2638A |
| 15 | M23-LST CDS | S. simulans |
| 16 | M23-LST PRT | S. simulans |
| 17 | Ami2638 CDS | Bacteriophage 2638A |
| 18 | Ami2638 PRT | Bacteriophage 2638A |
| 19 | CHAPK_CHAPK_CWT-LST CDS | artificial construct |
| 20 | CHAPK_CHAPK_CWT-LST PRT | artificial construct |
| 21 | M23-LST_M23-LST_CWT-LST CDS | artificial construct |
| 22 | M23-LST_M23-LST_CWT-LST PRT | artificial construct |
| 23 | Ami2638_ami2638_CWT-LST CDS | artificial construct |
| 24 | Ami2638_ami2638_CWT-LST PRT | artificial construct |
| 25 | HXaAmi2638_CBD2638 CDS | artificial construct |
| 26 | HXaAmi2638_CBD2638 PRT | artificial construct |
| 27 | HXaAmi2638_CWT-LST CDS | artificial construct |
| 28 | HXaAmi2638_CWT-LST PRT | artificial construct |
| 29 | HXaAmi2638_CWT-NM3 CDS | artificial construct |
| 30 | HXaAmi2638_CWT-NM3 PRT | artificial construct |
| 31 | HXaCHAPK_CBD2638 CDS | artificial construct |
| 32 | HXaCHAPK_CBD2638 PRT | artificial construct |
| 33 | HXaCHAPK_CWT-LST CDS | artificial construct |
| 34 | HXaCHAPK_CWT-LST PRT | artificial construct |
| 35 | HXaCHAPK_CWT-NM3 CDS | artificial construct |
| 36 | HXaCHAPK_CWT-NM3 PRT | artificial construct |
| 37 | HXaCHAPTw_CBD2638 CDS | artificial construct |
| 38 | HXaCHAPTw_CBD2638 PRT | artificial construct |
| 39 | HXaCHAPTw_CWT-LST CDS | artificial construct |
| 40 | HXaCHAPTw_CWT-LST PRT | artificial construct |
| 41 | HXaCHAPTw_CWT-NM3 CDS | artificial construct |
| 42 | HXaCHAPTw_CWT-NM3 PRT | artificial construct |
| 43 | HXaM23-LST_CBD2638 CDS | artificial construct |
| 44 | HXaM23-LST_CBD2638 PRT | artificial construct |
| 45 | HXaM23-LST_CWT-LST CDS | artificial construct |
| 46 | HXaM23-LST_CWT-LST PRT | artificial construct |
| 47 | HXaM23-LST_CWT-NM3 CDS | artificial construct |
| 48 | HXaM23-LST_CWT-NM3 PRT | artificial construct |
| 49 | HXaAmi2638_Ami2638_CBD2638 CDS | artificial construct |
| 50 | HXaAmi2638_Ami2638_CBD2638 PRT | artificial construct |
| 51 | HXaAmi2638_Ami2638_CWT-LST CDS | artificial construct |
| 52 | HXaAmi2638_Ami2638_CWT-LST PRT | artificial construct |
| 53 | HXaAmi2638_Ami2638_CWT-NM3 CDS | artificial construct |
| 54 | HXaAmi2638_Ami2638_CWT-NM3 PRT | artificial construct |
| 55 | HXaCHAPK_CHAPK_CBD2638 CDS | artificial construct |
| 56 | HXaCHAPK_CHAPK_CBD2638 PRT | artificial construct |
| 57 | HXaCHAPK_CHAPK_CWT-LST CDS | artificial construct |
| 58 | HXaCHAPK_CHAPK_CWT-LST PRT | artificial construct |
| 59 | HXaCHAPK_CHAPK_CWT-NM3 CDS | artificial construct |
| 60 | HXaCHAPK_CHAPK_CWT-NM3 PRT | artificial construct |
| 61 | HXaCHAPTw_CHAPTw_CBD2638 CDS | artificial construct |
| 62 | HXaCHAPTw_CHAPTw_CBD2638 PRT | artificial construct |
| 63 | HXaCHAPTw_CHAPTw_CWT-LST CDS | artificial construct |
| 64 | HXaCHAPTw_CHAPTw_CWT-LST PRT | artificial construct |
| 65 | HXaCHAPTw_CHAPTw_CWT-NM3 CDS | artificial construct |
| 66 | HXaCHAPTw_CHAPTw_CWT-NM3 PRT | artificial construct |
| 67 | HXaM23-LST_M23-LST_CBD2638 CDS | artificial construct |
| 68 | HXaM23-LST_M23-LST_CBD2638 PRT | artificial construct |
| 69 | HXaM23-LST_M23-LST_CWT-LST CDS | artificial construct |
| 70 | HXaM23-LST_M23-LST_CWT-LST PRT | artificial construct |
| 71 | HXaM23-LST_M23-LST_CWT-NM3 CDS | artificial construct |
| 72 | HXaM23-LST_M23-LST_CWT-NM3 PRT | artificial construct |
| 73 | His-tag with linker CDS | artificial construct |
| 74 | His-tag with linker PRT | artificial construct |
| 75 | LST CDS | S. simulans |
| 76 | LST PRT | S. simulans |
| 77 | HXaCHAP11_M23-2638_Ami2638_CBD2638 CDS | artificial construct |
| 78 | HXaCHAP11_M23-2638_Ami2638_CBD2638 PRT | artificial construct |
| 79 | HXaAmi11_M23-2638_Ami2638_CBD2638 CDS | artificial construct |
| 80 | HXaAmi11_M23-2638_Ami2638_CBD2638 PRT | artificial construct |
| 81 | HXaCHAPTw_Ami2638_M23-LST_CBD2638 CDS | artificial construct |
| 82 | HXaCHAPTw_Ami2638_M23-LST_CBD2638 PRT | artificial construct |
| 83 | HXaM23-LST_Ami2638_CBD2638 CDS | artificial construct |
| 84 | HXaM23-LST_Ami2638_CBD2638 PRT | artificial construct |
| 85 | HXaM23-2638_Ami2638_CBD2638_CBD2638 CDS | artificial construct |
| 86 | HXaM23-2638_Ami2638_CBD2638_CBD2638 PRT | artificial construct |
| 87 | HXaM23-2638_CBD2638 CDS | artificial construct |
| 88 | HXaM23-2638_CBD2638 PRT | artificial construct |
| 89 | HXaPly2638-Ply2638 CDS | artificial construct |
| 90 | HXaPly2638-Ply2638 PRT | artificial construct |
| 91 | HXaCHAPTw_Ami2638_M23-LST_CWT-LST CDS | artificial construct |
| 92 | HXaCHAPTw_Ami2638_M23-LST_CWT-LST PRT | artificial construct |
| 93 | HXaLST_LST CDS | artificial construct |
| 94 | HXaLST_LST PRT | artificial construct |
| 95 | HXaGFP_CBD2638 CDS | artificial construct |
| 96 | HXaGFP_CBD2638 PRT | artificial construct |
| 97 | CHAPΦ11 CDS | S. aureus phage phi 11 |
| 98 | CHAPΦ11 PRT | S. aureus phage phi 11 |
| 99 | AmiΦ11 CDS | S. aureus phage phi 11 |
| 100 | AmiΦ11 PRT | S. aureus phage phi 11 |

1. A method of treatment comprising the administration of a composition comprising a first and a second compound, wherein said first compound is an anti-inflammatory compound and said second compound is a compound specifically targeting a Staphylococcus and comprises one or more enzymatic active domains exhibiting target bond specificity an essential bond in a peptidoglycan layer of said Staphylococcus, and comprises at least one cell wall binding domain specifically binding the peptidoglycan cell wall of said Staphylococcus, and wherein said cell wall binding domain originates from or is a homologue of a Staphylococcus phage endolysin and/or an S. simulans lysostaphin and/or an S. capitis ALE-1 bacteriocin.

2. The method according to claim 1, wherein the first and the second compound are administered sequentially or simultaneously.

3. The method according to claim 1, wherein said one or more enzymatic active domains is selected from or is a combination of a domain of the group consisting of a cysteine, histidine dependent amidohydrolases/peptidase domain, an endopeptidase domain, an amidase domain and a glycosylhydrolase domain.

4. The method according to claim 1, wherein said second compound is a polypeptide that has at least 80% identity with SEQ ID NO: SEQ ID NO: 84; and/or said cell wall binding domain has at least 80% identity to any of SEQ ID NO: 6; and/or wherein said one or more enzymatic active domains has at least 80% identity to any of SEQ ID NO: 16.

5. The method according to claim 1, wherein said second compound is a recombinant polypeptide comprising a multiplicity of said one or more enzymatic active domains exhibiting target bond specificity.

6. The method according to claim 1, wherein said first compound is selected from the group consisting of a corticosteroid, a calcineurin inhibitor, an immunotherapeutic compound, a recombinant human IFN-gamma, a microbial probiotic, a cytokine modulator, an inflammatory cell recruitment blocker, and a T cell activation inhibitor.

7. The method according to claim 1, wherein the first compound is a corticosteroid in the range of 0.05 to 5% by weight of the total composition.

8. The method according to claim 7, wherein the first compound is a corticosteroid in the range of 0.05 to 2.5% by weight of the total composition.

9. The method according to claim 8, wherein the first compound is a corticosteroid in the range of 0.1 to 1% by weight of the total composition.

10. The method according to claim 1, wherein the patient is a human.

11. The method according to claim 1, wherein the composition is a topical formulation.


SEQUENCE LISTING (100 sequences)

SEQ ID NO: 1 - DNA, 1461 bp, Staphylococcus bacteriophage 2638A
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 2 - protein, 486 aa, Staphylococcus bacteriophage 2638A
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 3 - DNA, 279 bp, Staphylococcus simulans
tggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtga

SEQ ID NO: 4 - protein, 92 aa, Staphylococcus simulans
WKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 5 - DNA, 285 bp, Staphylococcus bacteriophage 2638A
tggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 6 - protein, 94 aa, Staphylococcus bacteriophage 2638A
WKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 7 - DNA, 282 bp, Staphylococcus bacteriophage phiNM3
ggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttt

SEQ ID NO: 8 - protein, 94 aa, Staphylococcus bacteriophage phiNM3
GKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 9 - DNA, 585 bp, Staphylococcus bacteriophage K
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaat

SEQ ID NO: 10 - protein, 195 aa, Staphylococcus bacteriophage K
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKN

SEQ ID NO: 11 - DNA, 420 bp, Staphylococcus bacteriophage Twort
atgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttatt

SEQ ID NO: 12 - protein, 140 aa, Staphylococcus bacteriophage Twort
MKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFI

SEQ ID NO: 13 - DNA, 438 bp, Staphylococcus bacteriophage 2638A
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagat

SEQ ID NO: 14 - protein, 146 aa, Staphylococcus bacteriophage 2638A
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKD

SEQ ID NO: 15 - DNA, 420 bp, Staphylococcus simulans
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatat

SEQ ID NO: 16 - protein, 140 aa, Staphylococcus simulans
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGY

SEQ ID NO: 17 - DNA, 510 bp, Staphylococcus bacteriophage 2638A
ggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggt

SEQ ID NO: 18 - protein, 169 aa, Staphylococcus bacteriophage 2638A
NKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDG

SEQ ID NO: 19 - DNA, 1407 bp, artificial
  artificial construct
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 20 - protein, 468 aa, artificial
  artificial construct
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 21 - DNA, 1209 bp, artificial
  artificial construct
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 22 - protein, 402 aa, artificial
  artificial construct
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 23 - DNA, 1686 bp, artificial
  artificial construct
gcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 24 - protein, 561 aa, artificial
  artificial construct
AKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 25 - DNA, 1131 bp, artificial
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 26 - protein, 376 aa, artificial
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 27 - DNA, 1125 bp, artificial
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 28 - protein, 374 aa, artificial
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 29 - DNA, 1131 bp, artificial
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 30 - protein, 376 aa, artificial
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 31 - DNA, 975 bp, artificial
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 32 - protein, 324 aa, artificial
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 33 - DNA, 912 bp, artificial
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 34 - protein, 303 aa, artificial
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 35 - DNA, 966 bp, artificial
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 36 - protein, 321 aa, artificial
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 37 - DNA, 927 bp, artificial
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 38 - protein, 308 aa, artificial
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 39 - DNA, 921 bp, artificial
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 40 - protein, 306 aa, artificial
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 41 - DNA, 927 bp, artificial
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 42 - protein, 308 aa, artificial
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 43 - DNA, 843 bp, artificial
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 44 - protein, 280 aa, artificial
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 45 - DNA, 837 bp, artificial
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 46 - protein, 278 aa, artificial
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 47 - DNA, 843 bp, artificial
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 48 - protein, 280 aa, artificial
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 49 - DNA, 1755 bp, artificial
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 50 - protein, 584 aa, artificial
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 51 - DNA, 1749 bp, artificial
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 52 - protein, 582 aa, artificial
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 53 - DNA, 1755 bp, artificial
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 54 - protein, 584 aa, artificial
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 55 - DNA, 1533 bp, artificial
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 56 - protein, 510 aa, artificial
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 57 - DNA, 1470 bp, artificial
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 58 - protein, 489 aa, artificial
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 59 - DNA, 1524 bp, artificial
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 60 - protein, 507 aa, artificial
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 61 - DNA, 1446 bp, artificial
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 62 - protein, 481 aa, artificial
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 63 - DNA, 1440 bp, artificial
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 64 - protein, 479 aa, artificial
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 65 - DNA, 1446 bp, artificial
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 66 - protein, 481 aa, artificial
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 67 - DNA, 1278 bp, artificial
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 68 - protein, 425 aa, artificial
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 69 - DNA, 1272 bp, artificial
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 70 - protein, 423 aa, artificial
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 71 - DNA, 1278 bp, artificial
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 72 - protein, 425 aa, artificial
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 73 - DNA, 63 bp, artificial
  artificial his-tag
atgagaggatcgcatcaccatcaccatcacggatctggctctggatctggtatcgagggaagg

SEQ ID NO: 74 - protein, 21 aa, artificial
  artificial Hist-tag
MRGSHHHHHHGSGSGSGIEGR

SEQ ID NO: 75 - DNA, 741 bp, Staphylococcus simulans
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtga

SEQ ID NO: 76 - protein, 246 aa, Staphylococcus simulans
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 77 - DNA, 2019 bp, artificial
  artificial construct
atgagaggatcgcatcaccatcaccatcacggatccatgcaagcaaaattaactaaaaatgagtttatagagtggttgaaaacttctgagggaaaacaattcaatgtggacttatggtatggatttcaatgctttgattatgccaatgctggttggaaagttttgtttggattacttctaaaaggtttaggtgcaaaagatattccgttcgctaacaacttcgacggattagctactgtataccaaaatacaccggacttcttagcacaacctggcgacatggtggtattcggtagcaactacggtgctggatatggtcacgttgcatgggtaattgaagcaactttagattacatcattgtatatgagcagaattggctaggcggtggctggactgacggaatcgaacaacccggctggggttgggaaaaagttacaagacgacaacatgcttatgatttccctatgtggtttatccgtccgaattttaaaagtgagacagcgccacgatcagttcaatctcctacacaagcacctaaaaaagaaacagctggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 78 - protein, 672 aa, artificial
  artificial construct
MRGSHHHHHHGSMQAKLTKNEFIEWLKTSEGKQFNVDLWYGFQCFDYANAGWKVLFGLLLKGLGAKDIPFANNFDGLATVYQNTPDFLAQPGDMVVFGSNYGAGYGHVAWVIEATLDYIIVYEQNWLGGGWTDGIEQPGWGWEKVTRRQHAYDFPMWFIRPNFKSETAPRSVQSPTQAPKKETAGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 79 - DNA, 2145 bp, artificial
  artificial construct
atgagaggatcgcatcaccatcaccatcacggatccaagccacaacctaaagcagtagaacttaaaatcatcaaagatgtggttaaaggttatgacctacctaagcgtggtagtaaccctaaaggtatagttatacacaacgacgcagggagcaaaggggcgactgctgaagcatatcgtaacggattagtaaatgcacctttatcaagattagaagcgggcattgcgcatagttacgtatcaggcaacacagtttggcaagccttagatgaatcacaagtaggttggcataccgctaatcaaataggtaataaatattattacggtattgaagtatgtcaatcaatgggcgcagataacgcgacattcttaaaaaatgaacaggcaactttccaagaatgcgctagattgttgaaaaaatggggattaccagcaaacagaaatacaatcagattgcacaatgaatttacttcaacatcatgccctcatagaagttcggttttacacactggttttgacccagtaactcgcggtctattgccagaagacaagcggttgcaacttaaagactactttatcaagcagattagggcgtacatggatggtaaaataccggttgccactgtctctaatgagtcaagcgcttcaagtaatacagttaaaccagttgcaagtgcaggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 80 - protein, 714 aa, artificial
  artificial construct
MRGSHHHHHHGSKPQPKAVELKIIKDVVKGYDLPKRGSNPKGIVIHNDAGSKGATAEAYRNGLVNAPLSRLEAGIAHSYVSGNTVWQALDESQVGWHTANQIGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNESSASSNTVKPVASAGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 81 - DNA, 1959 bp, artificial
  artificial construct
atgagaggatcgcatcaccatcaccatcacggatccatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 82 - protein, 652 aa, artificial
  artificial construct
MRGSHHHHHHGSMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 83 - DNA, 1554 bp, artificial
  artificial construct
atgagaggatcgcatcaccatcaccatcacggatccgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcttacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 84 - protein, 517 aa, artificial
  artificial construct
MRGSHHHHHHGSAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 85 - DNA, 1887 bp, artificial
  artificial construct
atgagaggatcgcatcaccatcaccatcacggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaagagctcggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 86 - protein, 628 aa, artificial
  artificial construct
MRGSHHHHHHGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKELGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 87 - DNA, 1035 bp, artificial
  artificial construct
atgagaggatcgcatcaccatcaccatcacggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgagctcggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 88 - protein, 344 aa, artificial
  artificial construct
MRGSHHHHHHGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGELGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 89 - DNA, 2961 bp, artificial
  artificial construct
atgagaggatcgcatcaccatcaccatcacggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaagagctcatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 90 - protein, 986 aa, artificial
  artificial construct
MRGSHHHHHHGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKELMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 91 - DNA, 1953 bp, artificial
  artificial construct
atgagaggatcgcatcaccatcaccatcacggatccatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 92 - protein, 650 aa, artificial
  artificial construct
MRGSHHHHHHGSMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 93 - DNA, 1521 bp, artificial
  artificial construct
atgagaggatcgcatcaccatcaccatcacggatccgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaaggagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtga

SEQ ID NO: 94 - protein, 506 aa, artificial
  artificial construct
MRGSHHHHHHGSAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 95 - DNA, 1143 bp, artificial
  artificial construct
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctcggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 96 - protein, 380 aa, artificial
  artificial construct
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 97 - DNA, 516 bp, Staphylococcus bacteriophage phi 11
atgcaagcaaaattaactaaaaatgagtttatagagtggttgaaaacttctgagggaaaacaattcaatgtggacttatggtatggatttcaatgctttgattatgccaatgctggttggaaagttttgtttggattacttctaaaaggtttaggtgcaaaagatattccgttcgctaacaacttcgacggattagctactgtataccaaaatacaccggacttcttagcacaacctggcgacatggtggtattcggtagcaactacggtgctggatatggtcacgttgcatgggtaattgaagcaactttagattacatcattgtatatgagcagaattggctaggcggtggctggactgacggaatcgaacaacccggctggggttgggaaaaagttacaagacgacaacatgcttatgatttccctatgtggtttatccgtccgaattttaaaagtgagacagcgccacgatcagttcaatctcctacacaagcacctaaaaaagaaacagct

SEQ ID NO: 98 - protein, 158 aa, Staphylococcus bacteriophage phi 11
MSIIMEVATMQAKLTKNEFIEWLKTSEGKQFNVDLWYGFQCFDYANAGWKVLFGLLLKGLGAKDIPFANNFDGLATVYQNTPDFLAQPGDMVVFGSNYGAGYGHVAWVIEATLDYIIVYEQNWLGGGWTDGIEQPGWGWEKVTRRQHAYDFPMWFIRP

SEQ ID NO: 99 - DNA, 642 bp, Staphylococcus bacteriophage phi 11
aagccacaacctaaagcagtagaacttaaaatcatcaaagatgtggttaaaggttatgacctacctaagcgtggtagtaaccctaaaggtatagttatacacaacgacgcagggagcaaaggggcgactgctgaagcatatcgtaacggattagtaaatgcacctttatcaagattagaagcgggcattgcgcatagttacgtatcaggcaacacagtttggcaagccttagatgaatcacaagtaggttggcataccgctaatcaaataggtaataaatattattacggtattgaagtatgtcaatcaatgggcgcagataacgcgacattcttaaaaaatgaacaggcaactttccaagaatgcgctagattgttgaaaaaatggggattaccagcaaacagaaatacaatcagattgcacaatgaatttacttcaacatcatgccctcatagaagttcggttttacacactggttttgacccagtaactcgcggtctattgccagaagacaagcggttgcaacttaaagactactttatcaagcagattagggcgtacatggatggtaaaataccggttgccactgtctctaatgagtcaagcgcttcaagtaatacagttaaaccagttgcaagtgca

SEQ ID NO: 100 - protein, 163 aa, Staphylococcus bacteriophage phi 11
NPKGIVIHNDAGSKGATAEAYRNGLVNAPLSRLEAGIAHSYVSGNTVWQALDESQVGWHTANQIGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMD